(19) United States
(12) Patent Application Publication
(10) Pub. No.: US 2022/0355066 A1
(43) Pub. Date: Nov. 10, 2022

(54) Dual-Lumen Catheter Assembly

(71) Applicant: Frances Benoist Compton, Dallas, TX (US)

(72) Inventor: Frances Benoist Compton, Dallas, TX (US)

(21) Appl. No.: 17/662,340

(22) Filed: May 6, 2022

Related U.S. Application Data
(60) Provisional application No. 63/185,343, filed on May 6, 2021.

(51) Int. Cl. A61M 25/00; A61M 25/06

(52) U.S. Cl. CPC A61M 25/003; A61M 25/0043; A61M 25/0631; A61M 1/3496

(57) ABSTRACT

A catheter assembly includes a shaft with two non-communicating lumens for simultaneous inflow and outflow of fluids from a vein. A return lumen extends to an outlet assembly at the tip. A draw lumen includes an inlet assembly, proximally of the outlet assembly, with multiple openings to avoid blockage causing pressure elevation due to suction. The draw lumen has a larger crescent-shape profile while the return lumen has a smaller, circular profile within the arms of the crescent shape.

### REFERENCE TO RELATED APPLICATIONS

This application claims priority to U.S. Provisional Patent Appl. No. 63/185,343 and incorporates that application in its entirety.

### FIELD OF THE INVENTION

In general, the present invention relates to a device, system or method including a catheter with two non-communicating lumens to allow for simultaneous inflow and outflow of fluids from a peripheral vein and a thinner shaft for a thinner needle insertion, for achieving access to peripheral veins (i.e., non-major veins with smaller diameters, such as the basilic vein). A single lumen, and a catheter with a single lumen, transports a fluid either towards or away from the body in one direction. A catheter with two lumens is advantageous to reduce the number of needle sticks, inserted needles and/or catheters, when fluids need to be transported towards (return) and away from (draw) the body simultaneously. In particular, fluids must be transported in such opposing directions in apheresis treatments. Apheresis treatment is a process that withdraws whole blood from the body, separates one or more components of the blood, and returns the remaining blood/fluid to the body, and is an example of an extracorporeal treatment process. In addition, in the draw process, it is important to both avoid, minimize, and/or reduce recirculation of from the return flow to the draw flow (i.e. mixing blood/fluids introduced by the return flow into the blood/fluids in the draw flow) and to avoid, minimize, and/or reduce high negative pressures (suction) around the point at which the draw is being made to ensure that the intended procedure is fulfilled without any complications (e.g., catheter collapse or adhesion to vessel wall resulting from elevated pressure from blocking the return). Recirculation occurrence is when the drawn fluid includes some fraction of fluid that was returning fluid. The recirculation occurrence between inflow and outflow of fluids must stay below 20% (meaning less than 20% of the drawn fluid was just previously returning fluid) and is ideally around 10% - 15% or even below 10%.

The catheter assembly is composed of a single peripheral venous catheter containing two separate, non-communicating, lumens that can transport fluids both to and from the body at the same time. The draw lumen removes blood from the body to be taken for extracorporeal treatment/removal. The draw lumen has an opening (or openings) into the vein which is more proximal to the return lumen opening. Multiple openings, which may be sized differently, may be used there to avoid/minimize draw pressure elevation resulting from a single draw lumen being entirely blocked. That causes a risk of that lumen adhering to the adjacent vessel wall and/or causing the catheter to collapse at high negative pressures. The return lumen brings treated blood and fluid back to the body. The return lumen has an opening (or openings) into the vein which is more distal to the draw lumen opening to decrease recirculation during continuous draw and return of fluids. The draw lumen is designed as a crescent shape being larger (cross-sectional area) (e.g. 18G) while the return lumen is designed as a smaller, circular shape (20G) within the arms of the crescent shape (i.e. with the crescent shape of the draw lumen wrapped around the circular shape of the return lumen).

The shaft of the catheter is meant to be inserted into a peripheral vein, and can allow for continuous, simultaneous outflow and inflow of fluids to and from the body for the purpose of extracorporeal treatment to include apheresis, dialysis, and other forms of blood treatment/removal. The ability to have simultaneous outflow and inflow of fluid from a patient through a single peripheral catheter is advantageous to patient care because it allows for improved access for different types of treatments and procedures. The catheter shaft, overall, is large enough to accommodate the draw lumen and return lumen wrapped around it, and is thus larger than either (e.g. 16G).

In another embodiment permitting greater flow but with a larger catheter, the draw lumen is 18G, the return lumen is 18G, and catheter shaft, overall, is 15G.

A needle is also used for gaining access to the vein, and is placed through catheter shaft to stiffen the shaft and provide a sharp end for entry. After entry, the catheter is advanced into the vein over the needle. The needle is used to enter the vein, and then is removed as catheter is advanced into the vessel, Then the needle is retracted and withdrawn safely once the catheter is in place. The hub of the needle will cover the opening of the return lumen in order to prevent anyone from trying to insert the separate return catheter while the needle is still in place (for safety reasons).

Flow rates of whole blood with an 18G lumen can reach as high as —150ml/min and 20G lumen can reach as high as ˜120ml/min, depending on the hematocrit of the blood (which alters the viscosity). Saline is able to run as high as 400ml/min through this device.

The invention described in this document details a novel and improved invention for a device and method of transporting fluids in opposing directions via a catheter including two lumens, as well as the insertion of such a catheter.

### BACKGROUND OF THE INVENTION

Flow rates of the blood and/or other fluids, both in respect to draw and return directions, are important. Apheresis flow rates max out at around 110 ml/min, whereas dialysis is often performed at flow rates as high as 400ml/min. Most apheresis procedures are run at a flow rate well under 100 ml/min. Thus, apheresis flow rates can range from about 60 ml/min. to about 100 or 110 ml/min. A key component in determining such flow is the size (internal size/cross-sectional area) of the lumens.

The gauge system is commonly used to measure the size of catheters, needles, tubes and tubing, and other devices in use in the medical community. A lower gauge number indicates a larger diameter. Catheters for peripheral veins, for example, exist in gauge sizes ranging from 14G to 26G. Table 1 shows, for needles, the nominal outer diameter (O.D.), inner diameter (I.D.), and wall thickness in mm for 14G-22G. The values of Table 1 are all for circular needles, but the reference to the gauge for a devices with a non-circular internal profile also implies a similar cross-sectional flow area as a circular devices of that gauge. Thus, for example, an 18G needle would have a nominal I.D. of 0.838mm and a cross-sectional flow area A=0.25π(0.838mm)²=0.552mm². An 18G catheter or needle with a non-circular internal profile would similarly have a cross-sectional flow area A=0.552mm².

TABLE 1

| Gauge | Nominal O.D. | Nominal I.D. | Nominal wall |
|---|---|---|---|
| (G) | (mm) | (mm) | (mm) |
| 14 | 2.108 ± 0.025 | 1.600 ± 0.076 | 0.254 ± 0.025 |
| 15 | 1.829 ± 0.013 | 1.372 ± 0.038 | 0.229 ± 0.013 |
| 16 | 1.651 ± 0.013 | 1.194 ± 0.038 | 0.229 ± 0.013 |
| 17 | 1.473 ± 0.013 | 1.067 ± 0.038 | 0.203 ± 0.013 |
| 18 | 1.270 ± 0.013 | 0.838 ± 0.038 | 0.216 ± 0.013 |
| 19 | 1.067 ± 0.013 | 0.686 ± 0.038 | 0.191 ± 0.013 |
| 20 | 0.9081 ± 0.0064 | 0.603 ± 0.019 | 0.1524 ± 0.0064 |
| 21 | 0.8192 ± 0.0064 | 0.514 ± 0.019 | 0.1524 ± 0.0064 |
| 22 | 0.7176 ± 0.0064 | 0.413 ± 0.019 | 0.1524 ± 0.0064 |

For drawing fluid from the body for apheresis, a desired minimum gauge size should be around 18G (having a 1.3 mm outer diameter). For returning fluid during apheresis, the minimum gauge size should be 20G (having about a 1.0 mm outer diameter). The draw lumen is larger to withstand the negative pressure which is generated with a high flow rate (as much as 150 ml/min.). A single draw lumen, having a larger size than each of a number of draw smaller lumens (having a same combined sized) can reduce the risk of damage to components of the drawn fluids, e.g. damage to red blood cells caused by caused by shearing forces (hemolysis). The return lumen may be smaller because it is used to push the fluid back to the patient and does not experience the same negative pressure. For apheresis procedures, a gauge size of 18G for the draw and 20G for the return provides an adequate lumen size to allow for the necessary whole blood flow rates.

Ideally, the needle and catheter shaft should not occupy more than 45% of the vein's diameter, which is considered a nursing standard. The catheter to vessel ratio (or CVR) of not more than 45% is required, where CVR represents the indwelling area consumed or occupied by an intravascular device inserted and positioned within the area of a venous or arterial blood vessel. In practice, the standard is evaluated by referencing the catheter to vessel ratio (or CVR tool) which uses catheter gauge/French (o.d.) and vessel diameter (i.d.) as inputs (and proxies for area) to identify combinations for which CVR is less than or equal to 33% (safe), over 33% and less than 45% (acceptable), or equal to or greater than 45% (unsafe). This tool was created to prevent CRT (Catheter-related thrombosis-blood clotting in the vein and is a complication of inserting central venous catheters (PICCs)). A 2016 practice standard recommended that the CVR can increase from 33% and now take up to 45% or less of the vessel diameter. They found that a CVR of <45% was satisfactory in risk prevention. The CVR chart consists of vessel size and catheter size on the x and y-axis respectively, where zones of safety are color-coded.

An apheresis machine, or other extracorporeal treatment device, will generate pressures as a means of conveying the blood or other fluids in and out of the body. The draw pressure supplied to the catheter from an external source is in the negative region. The return pressure applied to the catheter from the same external source will be in the positive region. Inlet pressure for apheresis is defined as the force per unit area on the blood flow as it is drawn from the patient. Similarly, return pressure for apheresis is defined as the force per unit area on the flow as it is returned to the patient. These are the pressures that are displayed on an apheresis machine.

As an example, for fluid flow at 100 ml/min through a 20G lumen with a radius of 0.50 mm into a human body with a pressure of 125 mmHg, the pressure in the lumen was found to be approximately 102 mmHg. This pressure is within the safe limit for the apheresis machine. These are the pressures in the catheter lumens as the blood is being drawn or returned. An apheresis machine, for example, has controls that display status ranges of pressure (green, yellow, or red). The corresponding pressure ranges for the green status are 0 to 182 mmHg and 0 to -182 mmHg for return and draw, respectively. For apheresis, green status is considered ideal, and procedures should not be run in the red. Overall, these status ranges will demonstrate if ideal pressure is being maintained.

### BRIEF STATEMENT OF THE INVENTION

An embodiment includes a double lumen peripheral catheter assembly capable of simultaneous draw and return of fluid in a peripheral vein. The catheter assembly (or parts thereof) may be manufactured using known methods and materials suitable for medical devices, which might include 3D-printed using stereolithography with a high resolution to accommodate finer details within the catheter, molding (e.g. by injection molding), and/or extrusion. Tubing and connectors may be standard items, such as threaded male/female connections (e.g. Luer connectors) and soft PVC medical tubing. The catheter shaft assembly portion of the assembly shaft with a 18G draw lumen for a draw flow (inflow of fluid drawn from the patient). In an embodiment, that 18G lumen begins around 20 millimeters from the distal end of the catheter and connects to a draw inlet assembly, comprising a number of holes connected to that 18G lumen. That number of holes may be two, three, four, or even six-eight. The shaft of the catheter shaft assembly portion also has a 20G return lumen for a return flow (outflow of fluid at the distal end of the catheter returning fluid to the patient) that connects to a return outlet assembly comprising one or more holes at and/or near the tip of the shaft assembly.

The shaft is rounded, for entry into a vein, and may be circular in transverse section, and have the same, or substantially the same diameter and profile along its length, except the tip which tapers to avoid vein tearing during entry/advancement. The shaft may be 16G (in the sense of its exterior dimensions), or have an O.D. of around 1.65 mm. The taper section may be about 0.78 mm in diameter. The catheter may be formed of suitable medical grade materials, including silicone or other polymers; in one embodiment, a polypropylene or PP-like resin may suffice; such a material may be translucent and flexible, e.g. with flexural modulus of 1,600 MPa.

In an embodiment, the return lumen may take a circular transverse profile while the draw lumen is a crescent-shaped or bean-shaped profile reflecting the return lumen and interlumen wall. In an embodiment, a circular wall around the return lumen coincides with all or a portion of the wall around the concave part of the crescent-shaped 18G draw lumen. Thus, between the lumens is an interlumen wall, formed internally to the shaft, that forms all or a portion of the concave exterior wall part of the crescent-shaped 18G draw lumen and forms the other part of the circular 20G return lumen. The outer circular wall of the shaft body surrounds the crescent-shaped 18G draw lumen and the circular 20G return lumen and coincides with all or a portion of the convex part of the exterior wall around the crescent-shaped 18G draw lumen and around the circular 20G return lumen. Because the draw lumen only extends for a portion of the length of the shaft, so does the interlumen wall. Thus, there is an interlumen wall between the draw lumen and the return lumen for a portion of the length of the shaft, which may be 16G, and the lumens are separate and non-communicating. The draw lumen terminates at the draw inlet assembly, meaning the shaft has a dual-lumen section, and a single-lumen section distal of the dual-lumen section, where the single-lumen section has the same, or substantially the same diameter and profile along its length as the dual-lumen section. The dual-lumen section includes the draw lumen and the return lumen, and the single-lumen section including the return lumen only.

In an embodiment, the return lumen may take a circular transverse profile while the draw lumen has an internal profile reflecting the return lumen and interlumen wall, such as circular, oval, semicircular, or similar shapes. In an embodiment, circular wall around the return lumen coincides with all or a portion of the wall around the 18G draw lumen. Thus, between the lumens is an interlumen wall, formed internally to the shaft, that forms all or a portion of the concave exterior wall part of the 18G draw lumen and forms the other part of the circular 20G return lumen. The outer circular wall of the shaft body surrounds the 18G draw lumen and the circular 20G return lumen and coincides with all or a portion of a part of the exterior wall around the 18G draw lumen and around the circular 20G return lumen. Because the draw lumen only extends for a portion of the length of the shaft, so does the interlumen wall. Thus, there is an interlumen wall between the draw lumen and the return lumen for a portion of the length of the shaft, which may be 15G or 16G, and the lumens are separate and non-communicating. The draw lumen terminates at the draw inlet assembly, meaning the shaft has a dual-lumen section, and a single-lumen section distal of the dual-lumen section, where the single-lumen section has the same, or substantially the same diameter and profile along its length as the dual-lumen section. The dual-lumen section includes the draw lumen and the return lumen, and the single-lumen section including the return lumen only.

The draw inlet assembly includes a plurality of holes connecting the exterior of the shaft and shaft assembly to the draw lumen. In an embodiment, there are three holes including a larger, primary (or main), inlet hole and two smaller, secondary inlet holes arranged in a staggered/triangular formation, e.g. that the secondary (or side) holes are longitudinally separated and radially-separate from the primary hole. Either may be proximal or distal. The purpose of the larger hole is to act as the origin point of inflow/draw from where fluid from the body is removed into the draw lumen by a main draw flow path. The purpose of the smaller holes is to relieve pressure the catheter may face and to prevent the collapse of the catheter walls on itself and to maintain maximum suction pressures within the acceptable range of 0 to −327 mmHg (or to 0 to −182 mmHg). The smaller holes provide a secondary flow path for the draw flow to enter the draw lumen, thus preventing or minimizing elevated suction pressures on the return lumen of the catheter. The primary holes may be about 1.0-1.5 mm in diameter, or about 1.0-1.5 mm by 2.5-3.0 mm in size, or about 1.24 mm by about 2.89 mm. The side holes may be 0.3-0.7mm, or about 0.52 mm or 0.5 mm, in diameter. The side holes have a longitudinal inlet spacing between themselves and the main hole. The nearer side hole is about 3.0-4.5 mm, about 3.74 mm, or about 4.5 mm, (edge-to-edge) from the primary hole, and about 1.0-2.0 mm, or about 1.48 mm, (edge-to-edge) from each another. The sides holes also have angular spacing or offset between themselves and the main hole. The side holes are offset about 30-55 degrees each, about 40-45 degrees each, or about 45 degrees each, in different directions from the primary hole. In an embodiment, a single, or additional, secondary holes may be provided, or a pair of primary holes of the same or approximately the same size may be provided.

The return outlet assembly includes one or more holes connecting the exterior of the shaft and shaft assembly to the return lumen. In an embodiment, there is one hole, located distally at the tip of the shaft and shaft assembly, which may be fully or substantially circular in shape. In an embodiment, there is one main hole, located distally at the tip of the shaft and shaft assembly, which may be fully or substantially circular in shape, as well as three extra fenestrations (which may be oval) located proximally of the main hole. Those extra fenestrations may be located distally at about 2.0-6.0 mm, or at about 3.0-4.0 mm, of the main hole, and are spaced evenly radially, at about 120-degree spacing. In an embodiment, there is one main hole, located distally at the tip of the shaft and shaft assembly, which may be fully or substantially circular in shape, as well as two or four extra fenestrations (which may be oval) located proximally of the main hole. Those extra fenestrations may be located distally at about 2.0-6.0 mm, or at about 3.0-4.0 mm, of the main hole, and are spaced even angularly about the long axis.

The draw inlet assembly and return outlet assembly are spaced apart longitudinally. The longitudinal separation, or draw-return flow spacing, or draw/return spacing, between the openings to the respective lumens assures minimal/no recirculation occurs within the catheter. The draw inlet assembly is spaced at a longitudinal distance from the return outlet assembly. That spacing may be at least 20 mm, about or exactly 20 mm, about 18-22 mm, about 10-25 mm, or about 15-20 mm.

The catheter is connected to an external treatment machine (e.g. an extracorporeal treatment machine/apheresis device) with the help of tubes affixed with connectors (e.g. female threaded connectors, such as Luer connectors). These connectors ensure secure attachment of the catheter to the machine tubing and helps prevent leakage. A sliding clamp to control flow rates in the tubes may be provided, and well as male threaded connectors (Luer caps) connected to the female threaded connectors to maintain sterility.

The catheter shaft assembly connects to the tubes and machine via a Y-body (or connector portion or divergent connector). The Y-body connects to the shaft assembly on its distal end via a shaft seat and includes a draw section and a return section. The draw section includes a Y-body draw lumen fluidly connecting a Y-body draw outlet (proximally) and a Y-body draw inlet (distally). The return section comprising a Y-body return lumen fluidly connecting a Y-body return inlet (proximally) and a Y-body return outlet (distally). The Y-body forms a divergent connector as it joins the shaft lumens (having combined circular profile) into a single body for delivery/return via a dual-lumen catheter, where the Y-body draw lumen diverges from the Y-body return lumen. The Y-body return lumen is aligned in a straight line with the return lumen of the catheter shaft to permit a rigid needle to be aligned in both. Thus, there are two separate openings on the proximal side of the divergent connector for connecting tubing and connectors, e.g. for needle/connection to the machine, and one combined connection with openings for the two lumens in the shaft assembly. This divergent connector permits simpler attachment of connectors and tubing, as well as removal of the needle during catheter insertion.

A retractable needle is placed through the circular return lumen of the catheter shaft via the proximal connectors and tubing for gaining access to the vein. The beveled end of the needle extends distally of the shaft and shaft assembly through return lumen outlet at the tip. Once the needle enters the vein, the catheter is advanced into the vein, over the stationary needle, so that both draw and return lumen openings are within the vein. Once the catheter is in place, the needle is retracted and withdrawn safely. A needle may be withdrawn directly out, or may be incorporated into a shielding safety device (or needle safety chamber) providing spring-loaded return or manual return to protect a user from the retracted needle. For a 20G return lumen, a 21G or 22G needle may be used, and may be long enough to extend from proximally of the proximal connectors and tubing to distally of the shaft and shaft assembly. A needle about 3-5 inches long may be used. A needle has a hub at its proximal end, which may be a simple hub to enclosing that end for handling, or may incorporate a flashback chamber to permit confirmation of vein access. A needle bevel may be matched to the angle formed by the tip of a catheter shaft.

In an embodiment, a dual-lumen catheter assembly includes the catheter, and two tubes connecting to one catheter hub each. One tube is the draw line tube and the other is the return line tube. Each tube is connected to a catheter hub on its proximal end for connection to other devices for return and drawing of fluids from the body.

In an embodiment, the catheter includes the Y-body and dual-lumen shaft assembly. The Y-body connects to the two tubes on the proximal end and to the shaft on the other, and serves to join the lines into a single body for delivery/return via a dual-lumen catheter. The shaft joins the Y-body at the shaft seat, which can be unitary with the shaft assembly or a structure to retain a non-unitary shaft assembly. The Y-body includes a draw section with a Y-body draw lumen fluidly connecting a Y-body draw outlet (proximally) and a Y-body draw inlet (distally). The Y-body includes a return section with a Y-body return lumen fluidly connecting a Y-body return inlet (proximally) and a y-body return outlet (distally). Preferably at least one of the Y-body draw lumen and Y-body return lumen diverges from the other to provide spacing between the Y-body return inlet and Y-body draw outlet to permit fitting of tubes or other connectors therein.

In an embodiment, the dual-lumen shaft assembly includes shaft having a generally o.d. of about 1.65 mm, a tip distally, a return lumen outlet at the tip, and a taper section adjacent to the tip tapering to about 0.78 mm. The shaft includes a shaft body forming walls about the exterior and an interlumen wall forming the dual-lumen section with draw lumen and return lumen, and with no interlumen wall forming the single-lumen section with return lumen only, which is distal of draw inlet assembly.

In an embodiment, the shaft assembly also includes a draw inlet assembly, formed of a plurality of inlet holes, and preferably of a main inlet and two side holes. Main inlet is about 1-1.5 mm by 2.5-3 mm in size, and side holes are about 0.3-0.7 mm by about 0.3-0.7 mm in size. Main inlet is preferably about 1.24 mm by about 2.89 mm in size, and side holes are about 0.52 mm by about 0.52 mm in size. Preferably the main inlet and the first side hole are separated by first inlet spacing (edge-to-edge) of about 3-4.5 mm, and the main inlet and the second side hole are separated by second (and different) inlet spacing (edge-to-edge) of about 5-6.5 mm, and the side holes are separated by third inlet spacing (edge-to-edge) of about 1-2 mm. More preferably the main inlet and the first side hole are separated by first inlet spacing (edge-to-edge) of about 3.74 mm, and the main inlet and the second side hole are separated by second (and different) inlet spacing (edge-to-edge) of about 5.74 mm, and the side holes are separated by third inlet spacing (edge-to-edge) of about 1.48 mm. Preferably, each side hole is separated from the main inlet angularly by about 30-55 degrees in different directions. More preferably, each side hole is separated from the main inlet angularly by about 40-45 degrees in different directions, and more preferably by about 45 degrees in different directions.

In an embodiment, the shaft body includes the return lumen (in the dual- and single-lumen sections) and the draw lumen (in the dual-lumen section). The shaft body also includes the shaft draw lumen outlet, fluidly connected by the draw lumen to the draw inlet assembly, and fluidly connecting the shaft assembly to the Y-body at the Y-body draw inlet. The shaft body also includes the shaft return inlet outlet, fluidly connected by the return lumen to the return lumen outlet, and fluidly connecting the shaft assembly to the Y-body at the Y-body return outlet. Preferably the draw/return spacing between the return lumen outlet and the draw inlet assembly is about 18-22 mm, and preferably at least about 20 mm.

In an embodiment, there is a dual-lumen catheter assembly, comprising a dual-lumen catheter shaft assembly having an exterior, comprising a distal tip; a draw lumen; a return lumen; and an interlumen wall between the draw lumen and the return lumen; a return outlet assembly connecting the return lumen to the exterior; and a draw inlet assembly connecting the draw lumen to the exterior; and wherein the return outlet assembly is adjacent to the tip; wherein the draw inlet assembly is proximal of the return outlet assembly; and wherein the draw inlet assembly comprises a plurality of inlet holes. Further embodiments include: the draw inlet assembly comprising a main inlet and a first side hole and a second side hole; the main inlet being substantially larger than said side holes; the main inlet and the first side hole separated by a first longitudinal inlet spacing of about 3.0-4.5 mm; the main inlet and the second side hole separated by a second longitudinal inlet spacing; and said first and second longitudinal inlet spacing being different. Further embodiments include: at least one of said side holes separated from the main inlet angularly by about 40-45 degrees; at least one of said side holes separated from the main inlet angularly by about 30-55 degrees; and each of said side holes separated from the main inlet angularly in different directions. Further embodiments include: the catheter shaft assembly further comprising: a 16G shaft body; the shaft body comprising the draw lumen and the return lumen; and wherein the draw lumen is at least 18G and the return lumen is at least 20G; and the shaft body further comprising: a dual-lumen section; and a single-lumen section distal of the dual-lumen section; wherein the draw lumen has a crescent-shaped profile and the return lumen has a circular profile. Further embodiments include: the catheter shaft assembly being flexible; and the draw lumen having a crescent-shaped profile and the return lumen having a circular profile. Further embodiments include: the catheter shaft assembly further comprising: a shaft body; the shaft body comprising exterior walls about the draw lumen and the return lumen; the shaft body comprising an interlumen wall between the draw lumen and the return lumen; wherein the draw lumen has a crescent-shaped profile and the return lumen has a circular profile. Further embodiments include: the catheter shaft assembly further comprising: a shaft body, comprising a dual-lumen section and a single-lumen section, the single lumen section being distal of the dual-lumen section; and a tapered section distal of the single-lumen section; and the distal tip being distal of the shaft body; and the single-lumen section having substantially the same diameter and profile along its length as the dual-lumen section; and the tapered section terminating in the distal tip. Further embodiments include: a Y-body proximal of the catheter shaft assembly; the Y-body comprising a draw section and a return section; the draw section comprising a Y-body draw lumen fluidly connected to the draw lumen of the catheter shaft assembly; the return section comprising a Y-body return lumen fluidly connected to the return lumen of the catheter shaft assembly; wherein the Y-body return lumen is aligned in a straight line with the return lumen of the catheter shaft assembly to permit a needle to be inserted inside both; and a retractable needle inserted within the Y-body return lumen and the return lumen of the catheter shaft assembly. Further embodiments include: the return outlet assembly comprising: a main hole; and a plurality of extra fenestrations; the main hole being located at the distal tip; and the extra fenestrations being located proximally of the main hole; and the extra fenestrations being located about 2-6 mm distally of the main hole. Further embodiments include: a draw/return spacing between the return outlet assembly and the draw inlet assembly of about 18-22 mm and draw/return spacing between the return outlet assembly and the draw inlet assembly of at least about 20 mm. Further embodiments include: a shaft body, the shaft body comprising a dual-lumen section; and a single-lumen section, the single lumen section being distal of the dual-lumen section; and wherein the single-lumen section has substantially the same diameter and profile along its length as the dual-lumen section; and the draw lumen having a crescent-shaped profile and the return lumen having a circular profile; and the draw inlet assembly further comprising a main inlet and a first side hole and a second side hole; the main inlet and the first side hole separated by a first longitudinal inlet spacing; the main inlet and the second side hole separated by a second longitudinal inlet spacing; and said first and second longitudinal inlet spacing being different; and each of said side holes separated from the main inlet angularly in different directions.

In an embodiment, there is a method of using a catheter assembly for draw and return of fluids from a vein, comprising: drawing fluids from an exterior of a dual lumen catheter shaft assembly via a plurality of inlet holes of a draw inlet assembly connecting a draw lumen to the exterior; and returning fluids to the exterior of the catheter shaft assembly via a return outlet assembly connecting a return lumen to the exterior; the returning step occurring adjacent to a distal tip of the catheter assembly; and the drawing step occurring proximally of said return step. Further embodiments include: maintaining recirculation of the fluids below 20%; maintaining recirculation of the fluids between about 10%-15%; maintaining recirculation of the fluids below 10%; and carrying out an apheresis treatment on the fluids after the drawing step and before the returning step. Further embodiments include: the catheter assembly comprising a 16G shaft body; the shaft body comprising the draw lumen and the return lumen; and wherein the draw lumen is at least 18G and the return lumen is at least 20G; the draw inlet assembly further comprising a main inlet and a first side hole and a second side hole; the main inlet and the first side hole separated by a first longitudinal inlet spacing; the main inlet and the second side hole separated by a second longitudinal inlet spacing; and said first and second longitudinal inlet spacing being different; and at least one of said side holes separated from the main inlet angularly by about 30-55 degrees. Further embodiments include: the draw lumen having a crescent-shaped profile and the return lumen having a circular profile; and the drawing step further comprising said drawing of fluids occurring at a draw/return spacing of about 18-22 mm. Further embodiments include: the catheter shaft assembly further comprising: a shaft body; the shaft body comprising exterior walls about the draw lumen and the return lumen; the shaft body comprising an interlumen wall between the draw lumen and the return lumen; wherein the draw lumen has a crescent-shaped profile and the return lumen has a circular profile; and retracting a retractable needle from Y-body return lumen of a Y-body and from the return lumen of the catheter shaft assembly; said Y-body return lumen aligned in a straight line with the return lumen of the catheter shaft assembly; and also advancing and Y-body and a Y-body return lumen of a Y-body and the catheter shaft assembly and the return lumen of the catheter shaft assembly over a retractable needle, said Y-body return lumen aligned in a straight line with the return lumen of the catheter shaft assembly. Further embodiments include: the catheter shaft assembly being flexible.

In an embodiment, there is a method of using a catheter assembly for draw and return of fluids from a vein, comprising: drawing fluids by a main draw flow path from the exterior of a dual-lumen catheter assembly into a draw lumen; the drawing step further comprising providing one or more secondary flow paths from the exterior to the draw lumen; and returning fluids to the exterior of the catheter assembly distally of the drawing step and adjacent to a distal tip of the catheter assembly. Further embodiments include: further comprising the step of maintaining recirculation of the fluids below 20%; comprising the step of maintaining recirculation of the fluids between 10% and 15%; and comprising the step of maintaining recirculation of the fluids below 10%. Further embodiments include: the catheter assembly comprising a 16G shaft body; the shaft body comprising the draw lumen and the return lumen; and wherein the draw lumen is at least 18G and the return lumen is at least 20G. Further embodiments include: the drawing step further comprising drawing fluids by: a first secondary flow path separated by a first inlet spacing from said the main flow path; and a second secondary flow path separated by a second inlet spacing from said the main flow path; said first and second inlet spacing being different. Further embodiments include: the drawing step further comprising drawing fluids by: a first secondary flow path separated angularly by about 30-55 degrees from said the main flow path; and a second secondary flow path separated angularly by about 30-55 degrees in the opposite direction from said the main flow path. Further embodiments include: the draw lumen having a crescent-shaped profile and the return lumen having a circular profile; and the drawing step further comprising said drawing of fluids occurring at a draw/return spacing of about 18-22 mm.

### BRIEF DESCRIPTION OF THE FIGURES

FIG. 1A is a left side view of an embodiment of a catheter assembly.

FIG. 1B is a top view of the device in FIG. 1A.

FIG. 2 is a front, left, top oblique view of an embodiment of a catheter assembly omitting the needle and some connector pieces.

FIG. 3A is a top view of a portion of an embodiment of a catheter assembly.

FIG. 3B is a left side view of the device in FIG. 3A.

FIG. 3C is a distal end view of the device in FIG. 3A.

FIG. 3D is a proximal end view of the device in FIG. 3A.

FIG. 3E shows a section view along line A—A from FIG. 3B.

FIG. 3F shows a section view along line B—B from FIG. 3B.

FIG. 3G shows a section view along line C—C from FIG. 3D.

FIG. 4A shows detail of a portion of an embodiment of a catheter shaft.

FIG. 4B shows detail of a portion of an embodiment of a catheter shaft.

FIG. 4C shows detail of a portion of an embodiment of a catheter shaft.

FIGS. 5A-5C show an embodiment of the process of vein access using an embodiment of a catheter assembly in a sectioned view.

FIG. 6 shows the blood flow in a vein with an embodiment of the catheter assembly in use after vein access.

FIG. 7A is a top view of a portion of an embodiment of a catheter assembly.

FIG. 7B is a left side view of the device in FIG. 7A.

FIG. 8 shows an embodiment of a catheter assembly in an embodiment of the process of vein access in a sectioned view.

FIG. 9A shows a section view of the shaft assembly of an alternative embodiment of a catheter assembly.

FIG. 9B shows a section view of the shaft assembly of an alternative embodiment of a catheter assembly.

FIG. 9C shows a section view of the shaft assembly of an alternative embodiment of a catheter assembly.

FIG. 10 shows steps of a process for carrying out an embodiment of the invention.

FIG. 11 shows steps of a process for carrying out an embodiment of the invention.

### DETAILED DESCRIPTION OF THE INVENTION

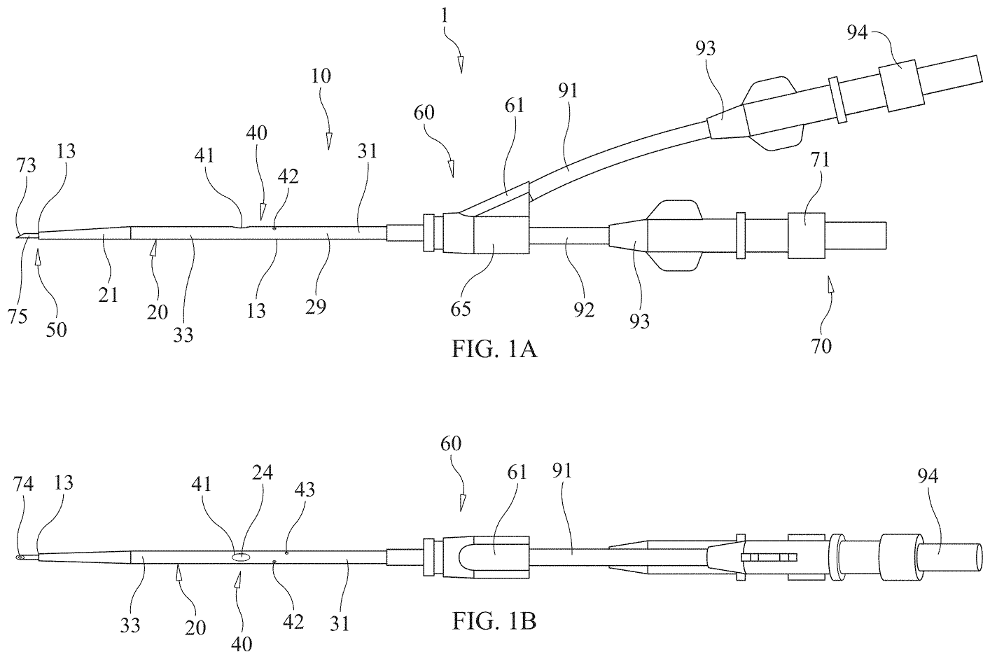

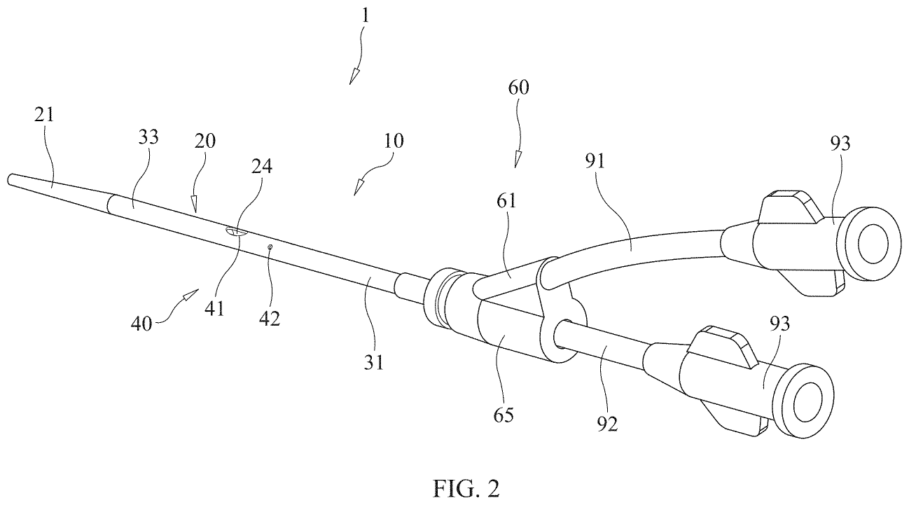

Referring to FIGS. 1A, 1B, 2, and 3A-3G, in an embodiment of the invention, as described in detail below, dual-lumen catheter assembly 1 includes dual-lumen catheter shaft 10, Y-body 60, and needle 70, as well as draw tubing 91, return tubing 92, female connectors 93, and male cap 94. Removing cap 94 and needle 70 permits connection to apheresis machine 96 (not depicted).

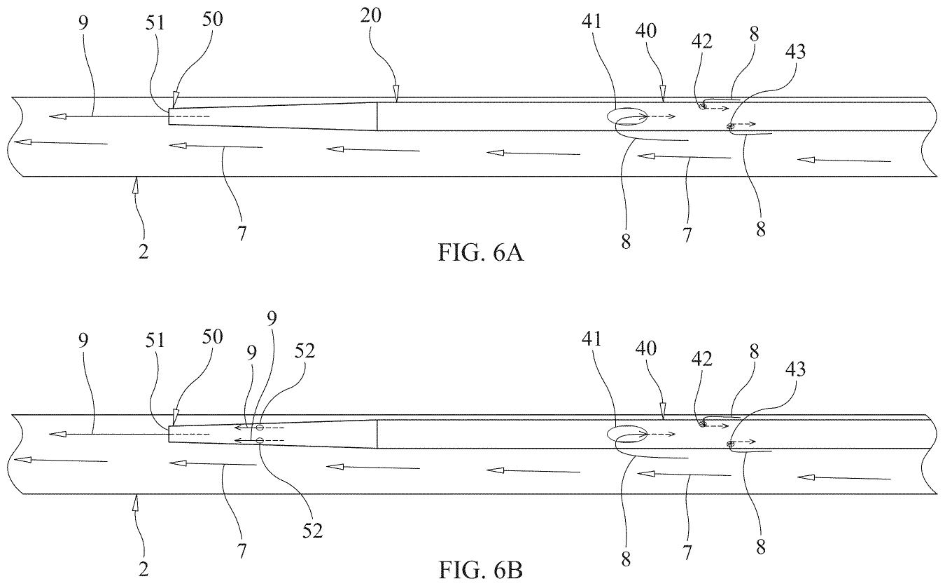

Dual-lumen catheter shaft 10 includes exterior 11, distal tip 13, shaft body 20, draw inlet assembly 40, and return outlet assembly 50. Exterior 11 of shaft 10 is, in use, exposed to interior 3 of vein 2 and fluid flow 7 of fluids 6 therein (see FIGS. 6A & 6B). Shaft body 20 is at the proximal end of dual-lumen catheter shaft 10, while distal tip 13 is at the distal end. Shaft body 20 includes tapered section 21 at its distal end, and proximal thereof, dual-lumen section 31, having circular or substantially circular profile 32, and proximal of dual-lumen section 31 single-lumen section 33, having circular or substantially circular profile 34. Shaft body 20 also includes, within dual-lumen section 31, draw inlet assembly 40, draw lumen 24 having crescent-shaped profile 24, and return lumen 26 having circular profile 27, and interlumen wall 28 therebetween, and exterior walls 29 therearound. Shaft body 20 also includes, within single-lumen section 31, return lumen 26 having circular profile 27 and exterior walls 29 therearound. Shaft body 20 also includes, within tapered section 21, return outlet assembly 50, and return lumen 26 therethrough.

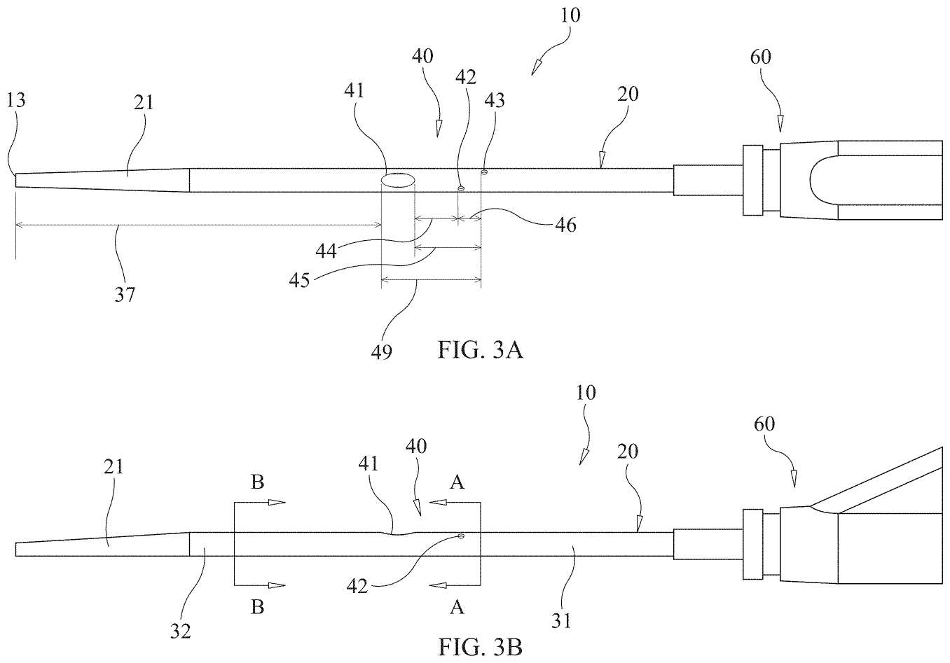

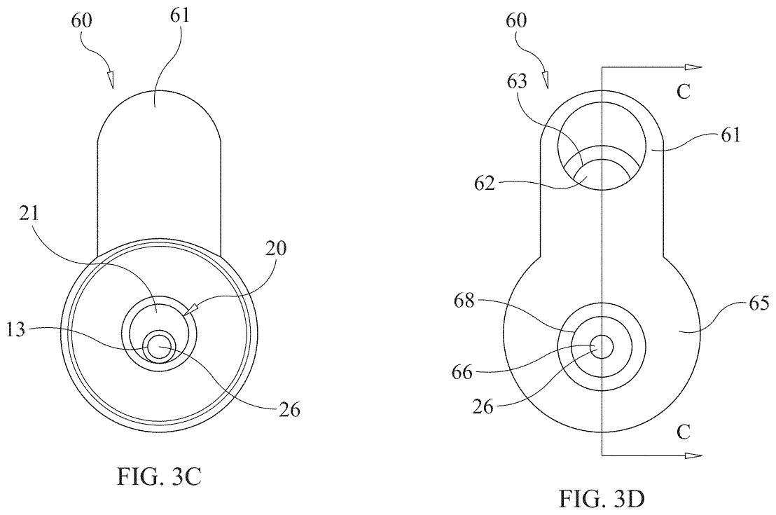

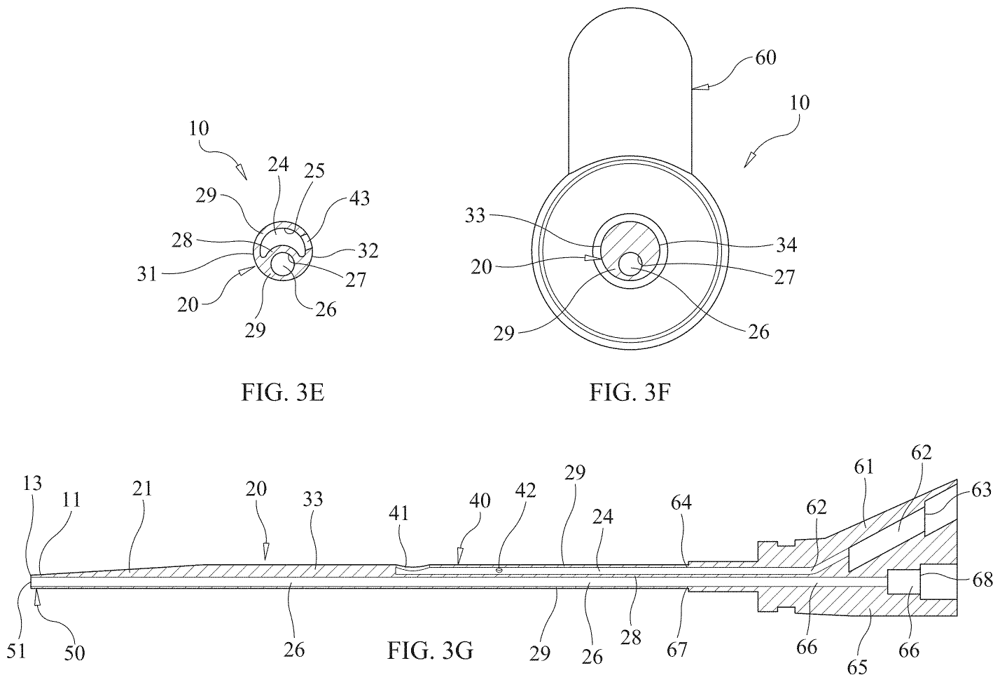

FIG. 3E shows, in section, crescent-shaped profile 25, circular profile 27, and interlumen wall 28. FIGS. 9A-9C show, in section, circular profile 27, and interlumen wall 28, with alternative profiles 25′, 25″ and 25′″.

Referring to FIGS. 1A, 1B, 2, and 3A-3G, in an embodiment, draw inlet assembly 40 includes main inlet 41, first side hole 42, and second side hole 43. Draw inlet assembly 40 is at the distal end of dual-lumen section of shaft body 20. Draw inlet assembly 40 has draw inlet assembly length 49.

Main inlet 41 may be about 1.0-1.5 mm in diameter, or about 1.0-1.5 mm by 2.5-3.0 mm in size, or about 1.24 mm by about 2.89 mm. Side holes 42, 43, may be 0.3-0.7mm, or about 0.52 mm or 0.5 mm, in diameter. Main inlet 41 connects draw lumen 24 to exterior 11 of dual-lumen catheter shaft assembly 10. Main inlet 41 forms a main draw flow path into draw lumen 24.

Side holes 42, 43 each have a longitudinal inlet spacing between themselves and main inlet 41. First side hole 42 has first longitudinal inlet spacing 44 of about 3.0-4.5 mm, about 3.74 mm, or about 4.5 mm, (edge-to-edge) from main inlet 41. Second side holes 43 has side hole spacing 46 of about 1.0-2.0 mm, or about 1.48 mm, (edge-to-edge) from first side hole 42, making second longitudinal inlet spacing 45 of about the sum of side hole spacing 46 and first longitudinal inlet spacing 44. Side holes 42, 43 each also have angular spacing or offset between themselves and main inlet 41. First side hole 42 has first angular separation 47 of about 30-55 degrees, about 40-45 degrees, or about 45 degrees from main inlet 41. Second side hole 43 has second angular separation 48 of about 30-55 degrees, about 40-45 degrees, or about 45 degrees from main inlet 41, but in the opposition direction of first angular separation 47. The three holes of draw inlet assembly thus form a roughly triangular formation. Side holes 42, 43 connect draw lumen 24 to exterior 11 of dual-lumen catheter shaft assembly 10. Side holes 42, 43 form secondary flow paths into draw lumen 24.

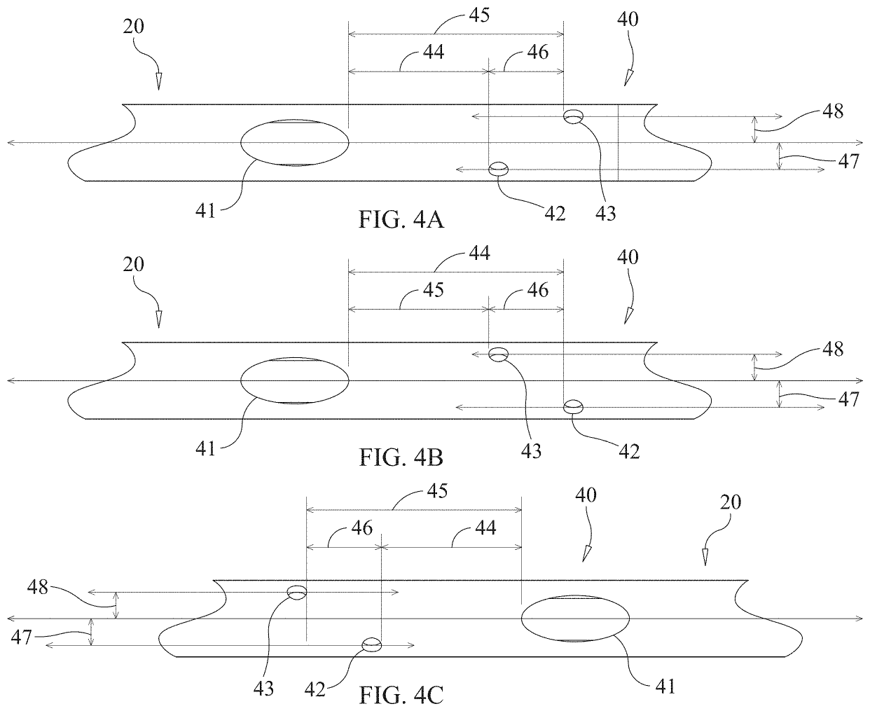

FIGS. 4A-4C show alternative arrangements of main inlet 41, first side hole 42, and second side hole 43, in which the closer of the side holes to main inlet 41 is different, or in which the distal/proximal positions of main inlet 41, on one hand, and first side hole 42 and second side hole 43, on the other, are switched.

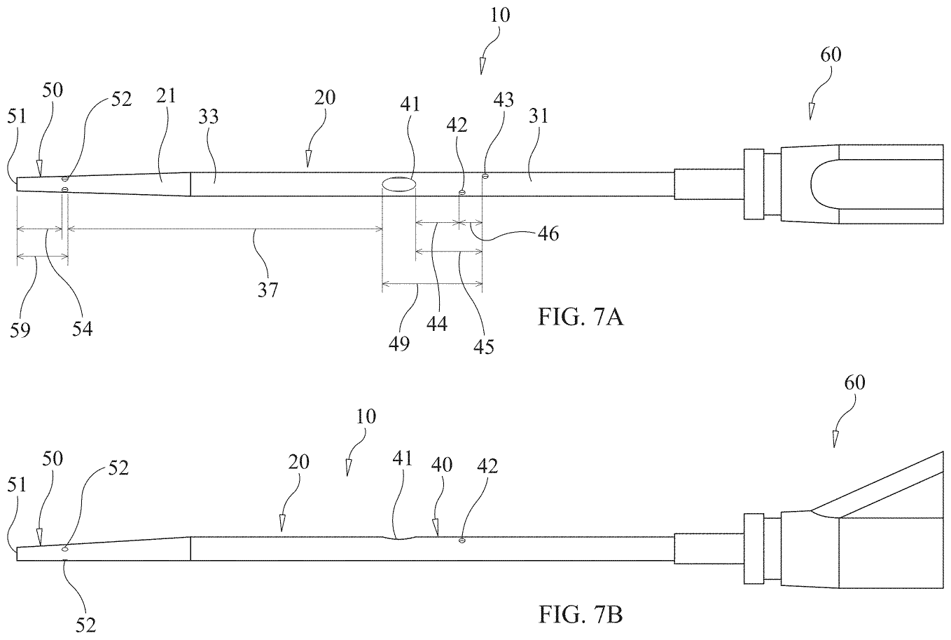

Returning to FIGS. 1A, 1B, 2, and 3A-3G, return outlet assembly 50 includes main hole 51 and, optionally, extra fenestrations 52 (see FIGS. 7A-7B). Return outlet assembly 50 has, where extra fenestrations 52 are provided, longitudinal outlet spacing 54 between main hole 51 and extra fenestrations 52, and has return outlet assembly length 59. Return outlet assembly 50 is adjacent to distal tip 13 of dual-lumen catheter shaft assembly 10, while main hole 51 is at distal tip 13. Main hole 51 faces distally and is at the distal end of return lumen 26, thus connecting return lumen 26 to exterior 11 of dual-lumen catheter shaft assembly 10.

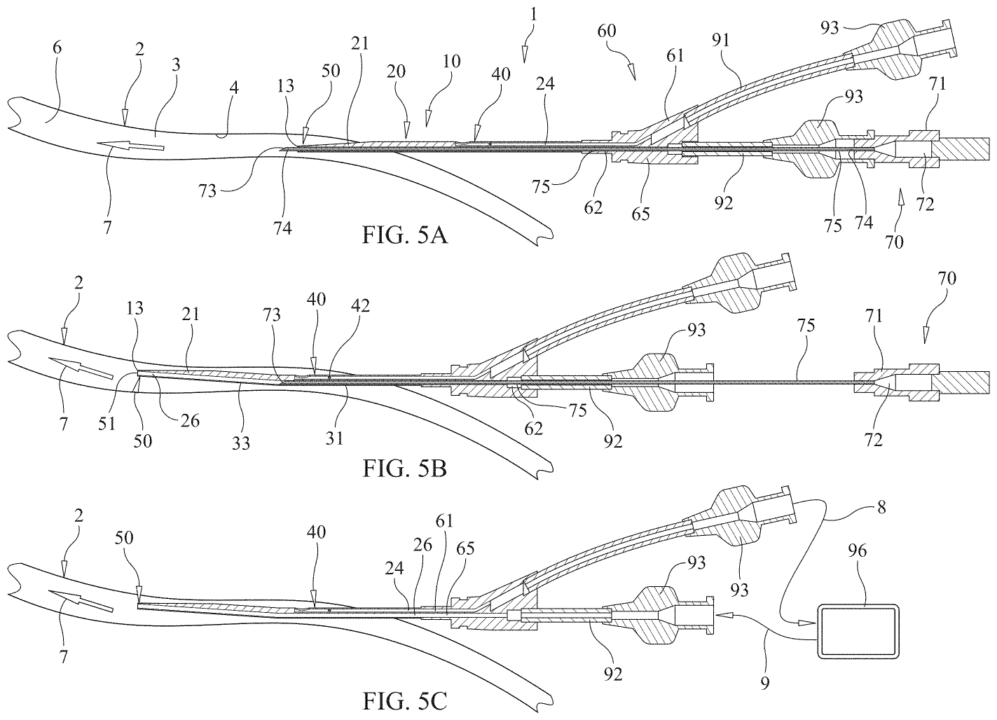

Dual-lumen catheter shaft assembly 10 connects to draw tubing 91 and return tubing 92 (and thence to apheresis machine 96) via Y-body 60 (or connector portion or divergent connector). Y-body 60 connects on its distal end to dual-lumen shaft assembly 10. Y-body 60 includes draw section 61 and return section 65. Draw section 61 includes Y-body draw lumen 62, Y-body draw outlet 63 (proximally), and Y-body draw inlet 64 (distally). Y-body draw lumen 62 fluidly connects Y-body draw inlet 64 (distally) and Y-body draw outlet 63 (proximally). Return section 65 includes Y-body return lumen 66, Y-body return outlet 67 (distally), and Y-body return inlet 68 (proximally). Y-body return lumen 66 fluidly connects Y-body return inlet 68 (proximally) and Y-body return outlet 67 (distally). Y-body 60 forms a divergent connector as it joins draw lumen 24 and return lumen 26, in shaft body 20, (having combined circular profile) into a single body for delivery/return to apheresis machine 96. In doing so, Y-body draw lumen 62 diverges from Y-body return lumen 66. Thus, there are two separate openings on the proximal side of the Y-body 60 for connecting tubing 91, 92 and connectors, e.g. for needle/connection to the machine, and one combined connection with openings for the two lumens in the shaft assembly. Y-body return lumen 66 is aligned in a straight line with return lumen 26 of shaft body 20 of dual-lumen catheter shaft 10 to permit rigid needle 70 to be aligned in both (see FIG. 1A & FIG. 5A with needle so aligned).

Turning to FIGS. 1A, 1B, 5A-5C, & 8, needle 70 includes hub 71, optionally flashback chamber 72, shaft 75, needle lumen 74, and beveled point 73. Needle lumen 74 runs down the interior of shaft 75, connecting flashback chamber to the exterior via an opening at beveled point 73. Hub 71 is used to safely handle the end of needle 70. Needle 70 may be 22G for for a 20G return lumen 26, where about a 130 mm needle may be long enough to extend from proximally of the proximal connector 93 to distally of distal tip 13.

Needle 70 is placed (before use) through return lumen 26 of dual-lumen catheter shaft 10 via Y-body return lumen 66 and connector 93 and tubing 92 for gaining access to interior 3 of vein 2. Beveled point 73 extends distally of shaft body 20 and distal tip 13 through main hole 51 of return lumen outlet assembly 50. Once needle 70 sufficiently enters vein 2 through wall 4 (in the direction against the direction of fluid flow 7), dual-lumen catheter shaft assembly 10 is advanced into vein 2, over needle 70 held stationary, until both draw inlet assembly 40 and return outlet assembly 50 are fully within vein 2. At this point, needle 70 has been partially retracted (by being stationary while assembly 10 advances). Once dual-lumen catheter shaft assembly 10 is in place, the rest of needle 70 is retracted and withdrawn safely. FIGS. 6A & 6B show the detail of two different embodiments of shaft body 20 inside vein 2, showing return flow 9 leaving return outlet assembly 50 and entering (joining) fluid flow 7 downstream of where draw flow 8 enters draw inlet assembly 40.

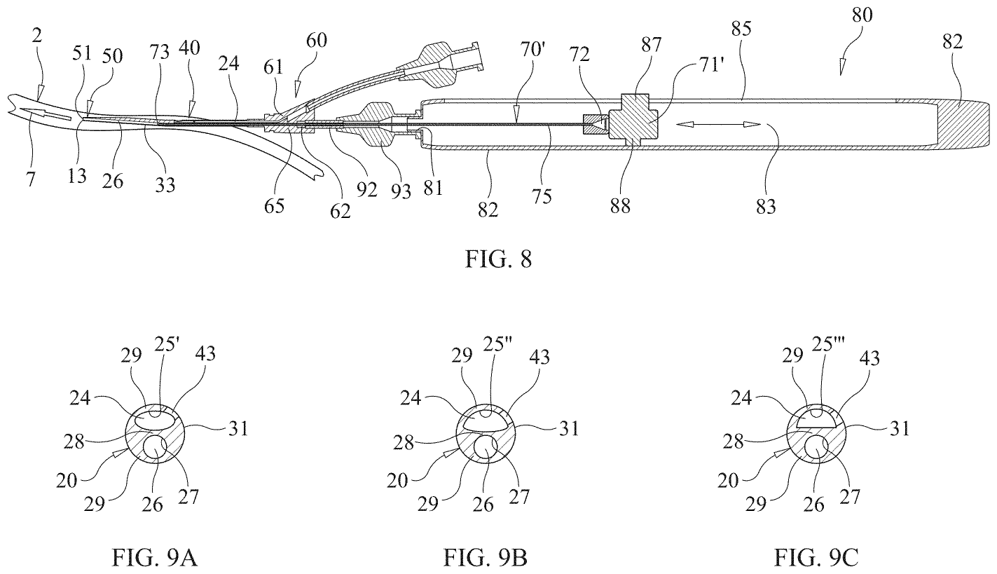

Needle 70 may be withdrawn directly or may be incorporated into shielding safety device 80 (see FIG. 8) to protect a user from the retracted needle. Shielding safety device 80 includes connector 81, casing 82, slot 85, and interior 83. A needle 70′ for using with shielding safety device 80 includes flashback chamber 72, shaft 75, as well as hub 71′ with, handle 87 and glider 88. Connector 81 is for engagement with connector 93 on return tubing 92, and permits all of shaft 75 to be extended and then be retracted therethrough. Handle 87 extends through slot 85 to permit a user to retract needle 70′, while glider 88 supports needle 70′ on interior 83 of casing 82.

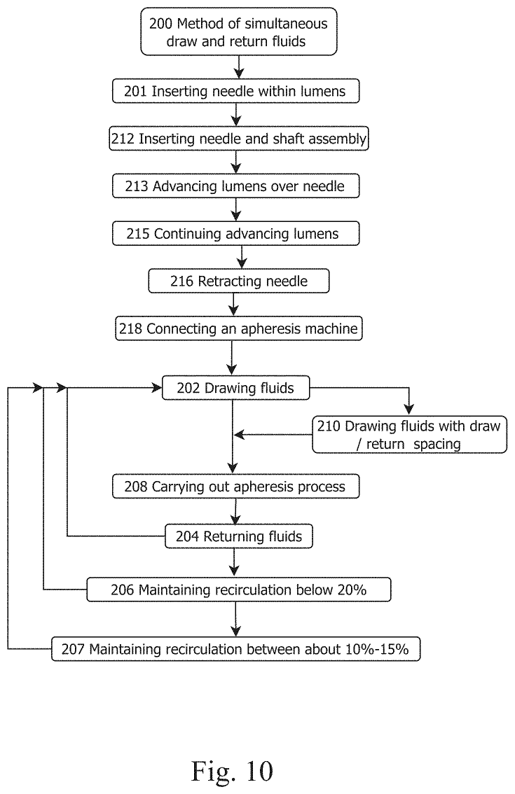

Turning to FIG. 10, an embodiment of the invention permits the simultaneous draw and return fluids from a vein via a single using a dual-lumen catheter assembly. The method 200 include the following steps. Step 201 is inserting a needle within a Y-body return lumen and a return lumen of catheter shaft assembly, the Y-body return lumen and a return lumen of catheter shaft assembly being aligned in a straight line, and with a beveled end of the needle extending distally of the dual-lumen catheter shaft assembly through an outlet at the tip. Step 212 is inserting the beveled end and the catheter shaft assembly into the vein sufficiently. Step 213 is advancing, distally, a return lumen of the catheter shaft assembly, and the catheter shaft assembly, and a Y-body return lumen of a Y-body, and the Y-body, over the shaft of the needle. Step 215 is continuing that advance until both a draw inlet assembly and a return outlet assembly are fully within the vein. Step 216 is retracting the needle from the return lumen of the catheter shaft assembly, and the catheter shaft assembly, and the Y-body return lumen of the Y-body, and the Y-body. Step 218 is connecting an apheresis machine via tubing and connectors on the proximal side of the Y-body. Step 202 is drawing fluids from an exterior of the dual lumen catheter shaft assembly via a plurality of inlet holes of a draw inlet assembly connecting a draw lumen to the exterior, the drawing step occurring proximally of return step 204. Step 204 is returning fluids to the exterior of the catheter shaft assembly via a return outlet assembly connecting a return lumen to the exterior; the returning step occurring adjacent to a distal tip of the catheter assembly. Step 206 is maintaining recirculation of the fluids below 20%. Step 207 is maintaining recirculation of the fluids between about 10%-15%. Step 208 is carrying out an apheresis treatment on the fluids after drawing step 202 and before returning step 204. Step 210 is drawing fluids at a draw/return spacing of about 18-22 mm.

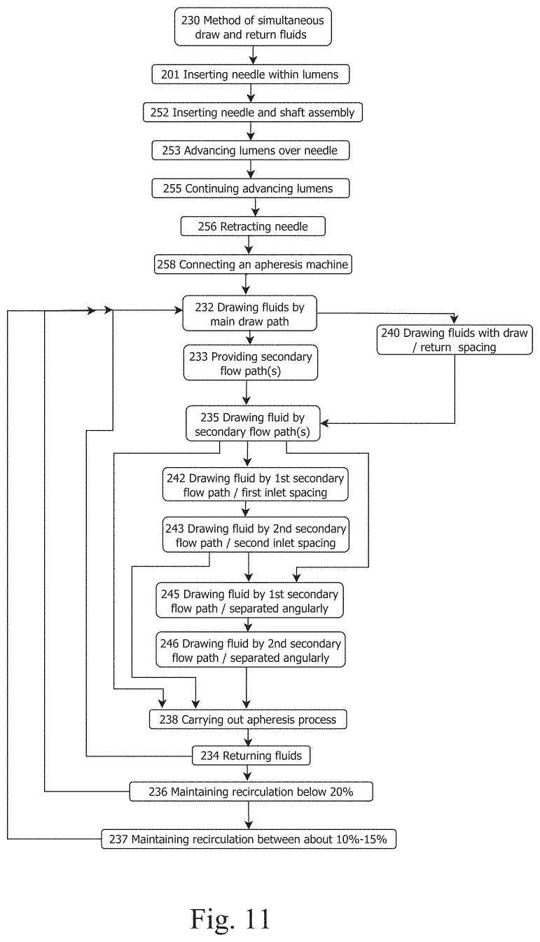

Turning to FIG. 11, an embodiment of the invention permits the simultaneous draw and return fluids from a vein via a single using a dual-lumen catheter assembly. The method 230 include the following steps. Step 251 is inserting a needle within a Y-body return lumen and a return lumen of catheter shaft assembly, the Y-body return lumen and a return lumen of catheter shaft assembly being aligned in a straight line, and with a beveled end of the needle extending distally of the dual-lumen catheter shaft assembly through an outlet at the tip. Step 252 is inserting the beveled end and the catheter shaft assembly into the vein sufficiently. Step 253 is advancing, distally, a return lumen of the catheter shaft assembly, and the catheter shaft assembly, and a Y-body return lumen of a Y-body, and the Y-body, over the shaft of the needle. Step 255 is continuing that advance until both a draw inlet assembly and a return outlet assembly are fully within the vein. Step 256 is retracting the needle from the return lumen of the catheter shaft assembly into a shielding safety device, and the catheter shaft assembly, and the Y-body return lumen of the Y-body, and the Y-body. Step 258 is connecting an apheresis machine via tubing and connectors on the proximal side of the Y-body. Step 232 is drawing fluids by a main draw flow path from the exterior of a dual-lumen catheter assembly into a draw lumen. Step 233 is providing one or more secondary flow paths from the exterior to the draw lumen. Step 235 is drawing fluids by one or more secondary flow paths from the exterior of a dual-lumen catheter assembly into a draw lumen. Step 234 is returning fluids to the exterior of the catheter assembly distally of the drawing step and adjacent to a distal tip of the catheter assembly. Step 236 is maintaining recirculation of the fluids below 20%. Step 237 is maintaining recirculation of the fluids between about 10%-15%. Step 238 is carrying out an apheresis treatment on the fluids after drawing step 232, providing step 233, and drawing step 235 and before returning step 234. Step 240 is drawing fluids at a draw/return spacing of about 18-22 mm. Step 242 is drawing fluids by a first secondary flow path separated by a first inlet spacing from said the main flow path. Step 243 is drawing fluids by a second secondary flow path separated by a second inlet spacing from said the main flow path, where the first and second inlet spacing are different. Step 245 is drawing fluids by a first secondary flow path separated angularly by about 30-55 degrees from the main flow path. Step 246 is drawing fluids by a second secondary flow path separated angularly by about 30-55 degrees in the opposite direction from the main flow path.

1. A dual-lumen catheter assembly, comprising:
a dual-lumen catheter shaft assembly having an exterior, comprising a distal tip;
a draw lumen;
a return lumen; and
an interlumen wall between the draw lumen and the return lumen;
a return outlet assembly connecting the return lumen to the exterior; and
a draw inlet assembly connecting the draw lumen to the exterior; and
wherein the return outlet assembly is adjacent to the tip;
wherein the draw inlet assembly is proximal of the return outlet assembly; and
wherein the draw inlet assembly comprises a plurality of inlet holes.

2. The catheter assembly of claim 1:
the draw inlet assembly comprising a main inlet and a first side hole and a second side hole.

3. The catheter assembly of claim 2, further comprising the main inlet being substantially larger than said side holes.

4. The catheter assembly of claim 2:
the main inlet and the first side hole separated by a first longitudinal inlet spacing of about 3.0-4.5 mm;
the main inlet and the second side hole separated by a second longitudinal inlet spacing; and
said first and second longitudinal inlet spacing being different.

5. The catheter assembly of claim 4, further comprising:
at least one of said side holes separated from the main inlet angularly by about 40-45 degrees.

6. The catheter assembly of claim 2, further comprising:
at least one of said side holes separated from the main inlet angularly by about 30-55 degrees.

7. The catheter assembly of claim 6, further comprising:
each of said side holes separated from the main inlet angularly in different directions.

8. The catheter assembly of claim 1, the catheter shaft assembly further comprising:
a 16G shaft body;
the shaft body comprising the draw lumen and the return lumen; and
wherein the draw lumen is at least 18G and the return lumen is at least 20G.

9. The catheter assembly of claim 8, the shaft body further comprising:
a dual-lumen section; and
a single-lumen section distal of the dual-lumen section;
wherein the draw lumen has a crescent-shaped profile and the return lumen has a circular profile.

10. The catheter assembly of claim 1, further comprising the catheter shaft assembly being flexible.

11. The catheter assembly of claim 1, the draw lumen having a crescent-shaped profile and the return lumen having a circular profile.

12. The catheter assembly of claim 1, the catheter shaft assembly further comprising:
a shaft body;
the shaft body comprising exterior walls about the draw lumen and the return lumen;
the shaft body comprising an interlumen wall between the draw lumen and the return lumen;
wherein the draw lumen has a crescent-shaped profile and the return lumen has a circular profile.

13. The catheter assembly of claim 1, the catheter shaft assembly further comprising:
a shaft body, comprising
a dual-lumen section; and
a single-lumen section, the single lumen section being distal of the dual-lumen section; and
a tapered section distal of the single-lumen section; and
the distal tip being distal of the shaft body.

14. The catheter assembly of claim 13, the single-lumen section having substantially the same diameter and profile along its length as the dual-lumen section.

15. The catheter assembly of claim 13, the tapered section terminating in the distal tip.

16. The catheter assembly of claim 1, further comprising:
a Y-body proximal of the catheter shaft assembly;
the Y-body comprising a draw section and a return section;
the draw section comprising a Y-body draw lumen fluidly connected to the draw lumen of the catheter shaft assembly;
the return section comprising a Y-body return lumen fluidly connected to the return lumen of the catheter shaft assembly;
wherein the Y-body return lumen is aligned in a straight line with the return lumen of the catheter shaft assembly to permit a needle to be inserted inside both.

17. The catheter assembly of claim 16, further comprising:
a retractable needle inserted within the Y-body return lumen and the return lumen of the catheter shaft assembly.

18. The catheter assembly of claim 1, the return outlet assembly comprising:
a main hole; and
a plurality of extra fenestrations;
the main hole being located at the distal tip; and
the extra fenestrations being located proximally of the main hole.

19. The catheter assembly of claim 18, the extra fenestrations being located about 2-6 mm distally of the main hole.

20. The catheter assembly of claim 1, further comprising a draw/return spacing between the return outlet assembly and the draw inlet assembly of about 18-22 mm.

21. The catheter assembly of claim 1, further comprising a draw/return spacing between the return outlet assembly and the draw inlet assembly of at least about 20 mm.

22. The catheter assembly of claim 1, further comprising:
a shaft body, the shaft body comprising
a dual-lumen section; and
a single-lumen section, the single lumen section being distal of the dual-lumen section; and
wherein the single-lumen section has substantially the same diameter and profile along its length as the dual-lumen section; and
the draw lumen having a crescent-shaped profile and the return lumen having a circular profile; and
the draw inlet assembly further comprising a main inlet and a first side hole and a second side hole;
the main inlet and the first side hole separated by a first longitudinal inlet spacing;
the main inlet and the second side hole separated by a second longitudinal inlet spacing; and
said first and second longitudinal inlet spacing being different; and
each of said side holes separated from the main inlet angularly in different directions.

23. A method of using a dual-lumen catheter assembly for draw and return of fluids from a vein, comprising:
drawing fluids from an exterior of a dual lumen catheter shaft assembly via a plurality of inlet holes of a draw inlet assembly connecting a draw lumen to the exterior; and
returning fluids to the exterior of the catheter shaft assembly via a return outlet assembly connecting a return lumen to the exterior;
the returning step occurring adjacent to a distal tip of the catheter assembly; and
the drawing step occurring proximally of said drawing step.

24. The method of claim 23, further comprising:
maintaining recirculation of the fluids below 20%.

25. The method of claim 23, further comprising:
maintaining recirculation of the fluids between about 10%-15%.

26. The method of claim 23, further comprising:
carrying out an apheresis treatment on the fluids after the drawing step and before the returning step.

27. The method of claim 23:
the catheter assembly comprising a 16G shaft body;
the shaft body comprising the draw lumen and the return lumen; and
wherein the draw lumen is at least 18G and the return lumen is at least 20G.

28. The method of claim 23, the draw inlet assembly further comprising a main inlet and a first side hole and a second side hole.

29. The method of claim 28, the main inlet and the first side hole separated by a first longitudinal inlet spacing;
the main inlet and the second side hole separated by a second longitudinal inlet spacing; and
said first and second longitudinal inlet spacing being different.

30. The method of claim 28, at least one of said side holes separated from the main inlet angularly by about 30-55 degrees.

31. The method of claim 23, the draw lumen having a crescent-shaped profile and the return lumen having a circular profile.

32. The method of claim 23, the drawing step further comprising said drawing of fluids occurring at a draw/return spacing of about 18-22 mm.

33. The method of claim 23, the catheter shaft assembly further comprising:
a shaft body;
the shaft body comprising exterior walls about the draw lumen and the return lumen;
the shaft body comprising an interlumen wall between the draw lumen and the return lumen;
wherein the draw lumen has a crescent-shaped profile and the return lumen has a circular profile.

34. The method of claim 23, further comprising:
advancing a Y-body return lumen of a Y-body and the return lumen of the catheter shaft assembly over a retractable needle;
said Y-body return lumen aligned in a straight line with the return lumen of the catheter shaft assembly.

35. The method of claim 23, further comprising the catheter shaft assembly being flexible.

36. A method of using a dual-lumen catheter assembly for draw and return of fluids from a vein, comprising:
drawing fluids by a main draw flow path from the exterior of a dual-lumen catheter assembly into a draw lumen;
the drawing step further comprising drawing fluids by one or more secondary flow paths from the exterior to the draw lumen; and
returning fluids to the exterior of the catheter assembly distally of the drawing steps and adjacent to a distal tip of the catheter assembly.

37. The method of claim 36, further comprising the step of maintaining recirculation of the fluids below 20%.

38. The method of claim 36:
the catheter assembly comprising a 16G shaft body;
the shaft body comprising the draw lumen and the return lumen; and
wherein the draw lumen is at least 18G and the return lumen is at least 20G.

39. The method of claim 36, the drawing step further comprising:
drawing fluids by a first secondary flow path separated by a first inlet spacing from the main flow path; and
drawing fluids by a second secondary flow path separated by a second inlet spacing from the main flow path;
said first and second inlet spacing being different.

40. The method of claim 36, the drawing step further comprising:
drawing fluids by a first secondary flow path separated angularly by about 30-55 degrees from the main flow path; and
drawing fluids by a second secondary flow path separated angularly by about 30-55 degrees in the opposite direction from the main flow path.

41. The method of claim 34, the draw lumen having a crescent-shaped profile and the return lumen having a circular profile.

42. The method of claim 34, the drawing step further comprising said drawing step occurring at a draw/return spacing of about 18-22 mm.
